(19) United States
(12) Patent Application Publication
(10) Pub. No.: US 2022/0305223 A1
(43) Pub. Date: Sep. 29, 2022

(54) Olfactory Delivery Device, System and Method for the Delivery of a Variety of Pharmaceutical Agents

(71) Applicant: Sharon K. Christie, Los Angeles, CA (US)

(72) Inventor: Sharon K. Christie, Los Angeles, CA (US)

(21) Appl. No.: 17/840,717

(22) Filed: Jun. 15, 2022

Related U.S. Application Data
(62) Division of application No. 16/229,066, filed on Dec. 21, 2018.
(60) Provisional application No. 62/610,445, filed on Dec. 26, 2017.

(51) Int. Cl. A61M 15/08; A61K 9/00; A61M 11/00; A61K 9/107

(52) U.S. Cl. CPC A61M 15/08; A61K 9/0043; A61M 11/005; A61K 9/107; A61M 2210/0618

(57) ABSTRACT

A nasal delivery system having a device that delivers a mist containing a pharmaceutical agent for delivery to a nasal cavity. The pharmaceutical agent in a liquid is in a micronized form with a particle size less than 5-microns that permeates a plurality of nasal membranes in a nasal cavity, crossing the blood brain barrier and entering the brain through cranial nerves. A route of administration of micronized liquid containing cannabinoids and terpenes using the device temporarily permeate the olfactory membranes to the olfactory and trigeminal nerves as well as permeate vascular rich olfactory pathway, thereby increasing diffusion of the therapeutic compounds into the cells that pass through the blood brain barrier for immediate effect, allowing inhalation while avoiding lung involvement. The device is manually controlled or preferably by an app on a mobile device.

### CROSS-REFERENCE TO RELATED APPLICATIONS

This application is a divisional nonprovisional utility application that is a division of the nonprovisional utility application, Ser. No. 16/229,066 filed in the United States Patent Office on Dec. 21, 2018 of the provisional patent application, Ser. No. 62/610,445, filed in the United States Patent Office on Dec. 26, 2017, and claims the priority thereof and is expressly incorporated herein by reference in its entirety.

### TECHNICAL FIELD

The present disclosure relates generally to a delivery device and a method of delivery of pharmaceutical agents. More particularly, the present disclosure relates to an olfactory delivery device and method for delivery of a variety of pharmaceutical agents for enhanced controlled precise nasal delivery.

### BACKGROUND

There are many routes of administration for pharmaceutical agents that are commonly used. The most common routes are oral and parenteral, such as intravenous and intramuscular.

In the instance of oral administration, problems arise if the patient has severe nausea, vomiting, problems swallowing or damage to the mouth, esophagus or stomach. Any absorption problems in the gastrointestinal system may result in less than optimal absorbance of the drug into the blood stream. Certain medications, such as insulin for example, cannot be administered orally because the gastrointestinal system decomposes the molecule or renders it inactive. These issues are often associated with patients undergoing chemotherapy with or without radiation for the treatment of cancer.

Intravenous administration results in distributing the drug systemically throughout the body rather than locally in a targeted organ. Intravenous and other parenteral means of administration are inherently medical procedures that produce pain, risk infection and other complications.

However, while less common, there are other routes such as inhalational, buccal, sublingual, suppository and topical that are less invasive and avoid gastrointestinal issues. Generally, these routes are appropriate when the nature of drug being administered requires these routes, or specific area or disease under treatment. Each route has inherent disadvantages for a patient who has an injury or compromised systems that a particular route requires. For example, using the inhalation route is contraindicated when the patient has damaged lungs.

Inhalation as a method of delivery with electronic cigarettes or “vape” pens with so called e-juice (flavored glycerin or propylene glycol) that produces a white cloud when heated. These ingredients are heated to their boiling points and create the cloud along with undesirable by-products that are harmful. Higher temperatures are required for the cannabis oils and distillates which again have potential harmful effects from the carbonation.

It is not uncommon for functionally active compounds to exist in nature, but lacking bioavailability. These compounds require assistance to provide better bioavailability to supply a myriad of benefits. As an example, turmeric is a flowering plant of the ginger family, Zingiberaceae, the roots of which are used in cooking. The plant contains the active curcumin but it is not optimally bioavailable when consumed orally. Evidence from numerous literatures reveals that curcumin has poor absorption, biodistribution, metabolism, and bioavailability. Continuous research on curcumin found some possible ways to overcome these problems to increase the bioavailability, longer circulation, better permeability, and resistance to metabolic processes of curcumin. Typically, these formulations have been prepared which include nanoparticles, liposomes, micelles, and phospholipid complexes as one example of a formulators' intervention to improve performance benefits.

In another example, the bioavailability of orally ingested THC ranges from only 6% to approximately 20% and the response time to experience is idiosyncratic and ranges from 1 hour to 3 hours depending on the individual, stomach contents, body mass as well as other environmental and genetic factors.

While these routes may be suitable for the particular purpose employed, or for general use, they would not be as suitable for the purposes of the present disclosure as disclosed hereafter.

In the present disclosure, where a document, act or item of knowledge is referred to or discussed, this reference or discussion is not an admission that the document, act or item of knowledge or any combination thereof was at the priority date, publicly available, known to the public, part of common general knowledge or otherwise constitutes prior art under the applicable statutory provisions; or is known to be relevant to an attempt to solve any problem with which the present disclosure is concerned.

While certain aspects of conventional technologies have been discussed to facilitate the present disclosure, no technical aspects are disclaimed and it is contemplated that the claims may encompass one or more of the conventional technical aspects discussed herein.

### BRIEF SUMMARY

The present disclosure provides a device and a method use, the device having a vapor guard configured for covering a nose so that a mist containing a pharmaceutical agent is delivered to a nasal cavity.

The device delivers a liquid containing a pharmaceutical agent in a micronized form that permeates a plurality of nasal membranes in a nasal cavity, crossing the blood brain barrier and entering the brain through cranial nerves. The device uses no heat and uses water as a co-solvent for formulated complex infusions of active phytocannabinoids as well as oil-soluble and water-soluble vitamins, terpenes and essential oils that when introduced to the apparatus, converts the infusion into a nano-emulsion by ultrasonic cavitation. Ultrasonic cavitation is the driving force for their formation and is mechanical instead of chemical, the ultrasonic cavitation providing a high shear force, able to break the infusion of oil into droplets the size of nanometers.

The device delivers a pharmaceutical agent regardless of its aqueous solubility. Accordingly, the ultrasonic technology incorporates very small amounts of a given active ingredient of a pharmaceutical agent irrespective of its polarity or solubility in water.

For example, the device delivers cannabinoids and terpenes to a patient without damage to the patient's lungs. Accordingly, the present disclosure provides a device for delivery of a micronized liquid containing cannabinoids and terpenes in certain ratios in appropriate vehicles to temporarily permeate the olfactory membranes, reaching the olfactory and trigeminal nerves in of the olfactory pathway, thereby increasing diffusion of the therapeutic compounds into the cells that pass through the blood brain barrier for immediate effect, allowing inhalation while avoiding lung involvement.

Accordingly, this disclosure presents a nasal delivery system having a device that delivers a mist containing a pharmaceutical agent to a nasal cavity and a method for using the same. The device changes the functionality or characteristics of a material during its use and in particular, when in use with phytocannabinoids such as cannabidiols such as CBD and cannabinols such as THC. The challenge in delivering a measurable bioavailable dose has to do with not only the method of delivery but the form in which the active is prepared so as to insure bioavailability. The challenge with the active ingredients found with in Cannabis sativa and related hemp plants is that the THC and cannabinoids such as CBD as well as other phytocannabinoids is their lack of water solubility or compatibility. These compounds are lipophilic and not hydrophilic. Formulation using a wide variety of adjuncts coupled with ultrasonic cavitation display hydrophilic properties, which may allow use in a wide array of formulations and pharmaceutical preparations.

The device provides ultrasonic technology to micronize the given active ingredient irrespective of its polarity or solubility in water. The device is manually controlled or preferably by an app on a mobile device.

The pharmaceutical agent in the liquid is in a micronized form with a particle size less than 5-microns that permeates a plurality of nasal membranes in a nasal cavity, entering the brain through cranial nerves, eliminating the necessity of crossing the blood-brain barrier. The route of nasal administration of the micronized liquid containing cannabinoids and terpenes uses the device, allowing the pharmaceutical agent to temporarily permeate the olfactory membranes to the olfactory and trigeminal nerves as well as permeate vascular rich olfactory pathway, thereby increasing diffusion of the therapeutic compounds into the cells that pass through the blood brain barrier, allowing inhalation while avoiding lung involvement.

This unique combination of technologies enables this device and method of use to enter the olfactory pathway, bypass the blood brain barrier with a “nose to brain” delivery and deliver a much smaller more controlled dose much less than any of the current methods of delivery. This “nose to brain” innovation delivers the active ingredient such as the THC, CBD or other lipophilic moiety directly to the receptor sites of the brain and neuro system resulting in a response within 15-30 seconds. Embodiments of the micronized pharmaceutical agents in the present disclosure display hydrophilic properties, which may allow use in a wide array of formulations and pharmaceutical preparations.

The present disclosure describes a nasal delivery system that is the logical choice for topical treatment of local diseases in the nose and paranasal sinuses such as allergic and non-allergic rhinitis and sinusitis. The nose is also considered an attractive route for needle-free vaccination and for systemic drug delivery, especially when rapid absorption and effect are desired. In addition, nasal delivery and olfactory delivery may help address issues related to poor bioavailability, slow absorption, drug degradation, and adverse events in the gastrointestinal tract and avoids the first-pass metabolism in the liver. However, when considering nasal delivery devices and mechanisms, it is important to keep in mind that the prime purpose of the nasal airway, and concurrently of nasal drug delivery, is to protect the delicate lungs from hazardous exposures such as by-products of combustion.

The present disclosure addresses at least one of the foregoing disadvantages of the prior art. However, it is contemplated that the present disclosure may prove useful in addressing other problems and deficiencies in a number of technical areas. Therefore, the claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

### BRIEF DESCRIPTION OF THE DRAWINGS

In the drawings, like elements are depicted by like reference numerals. The drawings are briefly described as follows.

FIG. 1 is a perspective view of an example embodiment of a nasal delivery device.

FIG. 2 is a side elevational view of the example embodiment of the nasal delivery device.

FIG. 3 is a sectional view of the side elevational view of FIG. 2 of the example embodiment of the nasal delivery device. The dotted line in FIG. 2 shows from where the sectional view was taken.

FIG. 4 is a diagrammatic view of the route of delivery to a brain by a nasal delivery device.

FIG. 5 is a flow chart diagram showing a method of controlling the delivery of a drug by a nasal delivery device.

FIG. 6 is a wireless network diagram showing the method of controlling the delivery of a drug by a nasal delivery device.

The present disclosure now will be described more fully hereinafter with reference to the accompanying drawings, which show various example embodiments. However, the present disclosure may be embodied in many different forms and should not be construed as limited to the example embodiments set forth herein. Rather, these example embodiments are provided so that the present disclosure is thorough, complete and fully conveys the scope of the present disclosure to those skilled in the art.

### DETAILED DESCRIPTION OF THE PREFERRED EMBODIMENTS

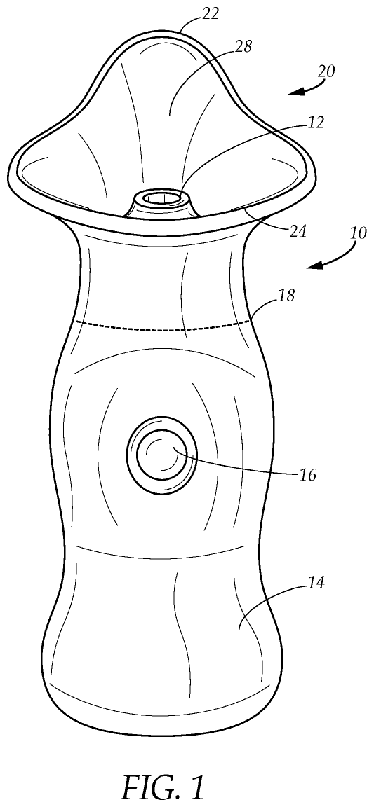

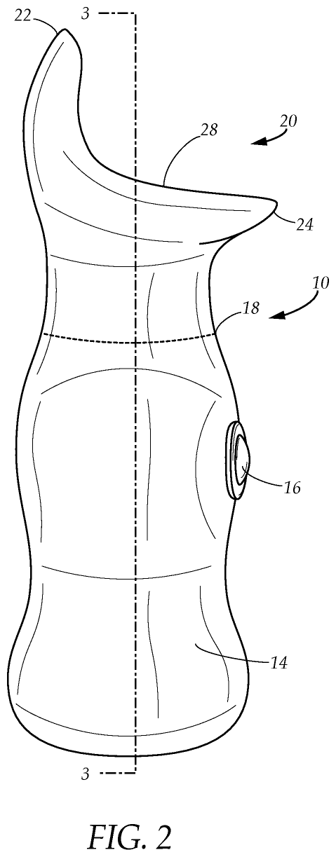

FIGS. 1-3 illustrate a device 10 for nasal delivery. The device 10 provides enhanced delivery of lipid and non-lipid molecules, both therapeutic and non-therapeutic compounds, into the cells utilizing the olfactory pathway and inhalation. Olfactory delivery has been shown to non-invasively deliver compounds from the nose to the brain in minutes along the olfactory and trigeminal nerve pathways, by passing the blood-brain barrier.

The device 10 has a vapor guard 20 atop a handle 14. The handle 14 has a switch 16 for turning the device 10 on and off. The handle 14 and the vapor guard 20 rotate with respect with to each other along a click closure 18.

The vapor guard 20 has a bowl 28 having a volume defined by a bridge guard 22 at a top 20T of the vapor guard, the bridge guard connecting to a nostril guard 24 at a bottom 20B. Inside the bowl 28 of the vapor guard 20 is a mist outlet 12.

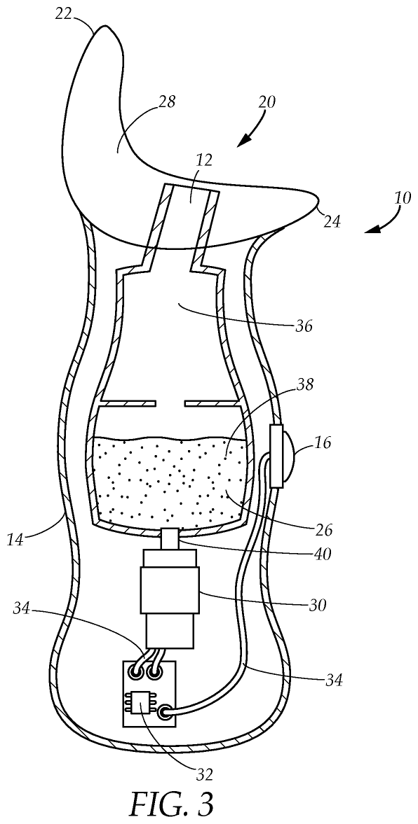

Referring to FIG. 3, inside the handle 14 is a reservoir 26 for holding a pharmaceutical agent in a liquid 38. The pharmaceutical agent may be dissolved, suspended or dispersed in the liquid 38. A motorized air pump 30 is operationally connected to the reservoir 26 through a tube 40. The pharmaceutical agent may be broadly interpreted to include nutritional, nutraceuticals, naturally occurring, semi-synthetic and synthetic medicinal compounds and other physiological active ingredients as non-limiting examples.

The device 10 provides ultrasonic technology through an acoustic vibrator to micronize the given active ingredient irrespective of its polarity or solubility in water.

In the example embodiment, the acoustic vibrator is the motorized air pump 30, using piezo-electricity to produce ultrasonic waves to atomize the liquid 38 in the reservoir, produces liquid particles that are less than 5 microns in size. In an additional example embodiment, the motor of the motorized air pump 30 provides acoustic waves to micronize the liquid. The reservoir 26 is fluidly connected to the mist outlet 12. The liquid particles enter a chamber 36 above the reservoir 26 and then are propelled through the mist outlet 12 into the bowl 28 of the vapor guard 20.

A controller 32 such as a printed circuit board or other similar technology known to people of ordinary skill in the art is operationally connected to the motorized air pump 30 and the switch 16 by wires 34.

The device 10 is intended to achieve superior bio-distribution, bioavailability, and decreased dose-to-dose variability of both small molecules and biologic drugs in patients when delivering the pharmaceutical agent nasally.

A patient places the pharmaceutical agent in liquid 38 by opening along the click closure 18 and filing the reservoir 26. In one example embodiment, the reservoir 26 is interchangeable, so that an empty reservoir is replaced with a pre-filled reservoir. The interchangeable reservoir may employ an ampule or cartridge in the pre-filled reservoir. In another example embodiment, the pre-filled reservoir has a metering closure such that a pre-measured dose is released from the reservoir.

The patient places the vapor guard 20 over his or her nose, the bridge guard 22 resting on the patient's nose bridge and the nostril guard 24 is placed under the nostrils. The vapor guard 20 completely encompasses the nose, creating a substantially sealed chamber around the nose. The device 10 may be handheld or rest on a table, freestanding or in a holder to stabilize the device, for hands-free operation.

The patient turns on the air pump motor 30, releasing microfluidized liquid particles less than 5 microns in size into the reservoir 26 and up through the mist outlet 12 into the bowl 28. The patient inhales the liquid particles into the olfactory pathway. The vapor guard 20 directs the microfluidized liquid into the upper nasal cavity without substantial leakage or loss of the pharmaceutical agent. The patient does not need to inhale deeply because the route of administration is not through the lungs.

It is understood by those of ordinary skill in the art these steps may be practiced directly by a patient or user, by the patient with the help of an assistant or health care worker.

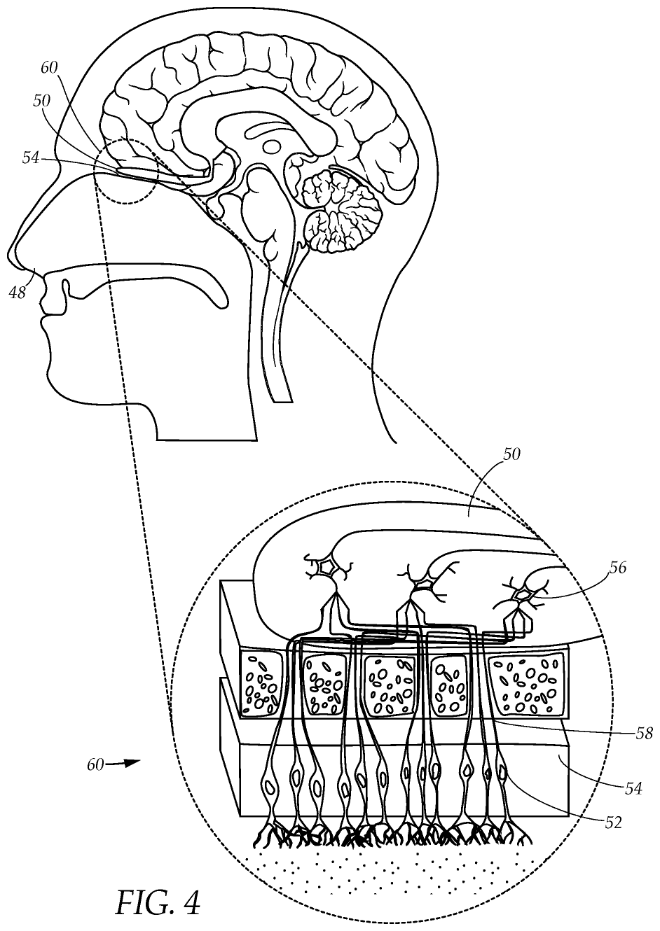

The device 10 as described hereinabove is designed to deliver drugs to the upper nasal cavity for improved bio-distribution. FIG. 4 shows the pathway of the pharmaceutical agent when inhaled. The pharmaceutical agent enters a nasal cavity 48 and into an olfactory region 60. The pharmaceutical agent binds to a plurality of receptor cells 52 located within an olfactory membrane 54 and are transmitted across a plurality of axons 58 of a plurality of neurons 56 located in an olfactory bulb 50. The neurons 56 are branches of an olfactory nerve. The olfactory nerve is the first cranial nerve and leads directly to the brain.

By delivering therapeutic pharmaceutical agents to the upper nasal cavity 48, the device takes advantage of the vascular rich olfactory region for improved bioavailability and has the potential to target the brain via the olfactory and trigeminal nerves. The trigeminal nerve is the fifth cranial nerve and has three major branches. One branch, the maxillary branch has neurons in the nasal cavity 48 and sinuses.

The nasal cavity 48 is a vastly underutilized entry point for therapeutics into the circulation. Compounds pass the blood brain barrier providing near immediate response in the central nervous system.

For most devices, the primary challenge in achieving significant nasal drug delivery is depositing drugs in the deeper regions of the nasal cavity 48. Due to the complex architecture of this area, drugs delivered with standard nasal devices such as droppers, sprays, or pumps typically deposit less than 5% of the compound in the olfactory region and are generally too high a dose to activate the olfactory pathway receptors which require a vapor phase micro dose to be activated. These other methods result in a large fraction of drug deposition in this region which can lead to direct greater uptake into the vasculature depending on the drug and requiring a longer time to activate and elicit the intended response.

The device 10 as described hereinabove overcomes these problems. The device 10 and a method of use requires a specially formulated pharmaceutical agent in a liquid. The pharmaceutical agent may be a lipid or a non-lipid. The compounds delivered may be, for example, but not limited to chemotherapeutic drugs, antibiotics, analgesics and anxiolytics. The present device and method of diffusion temporarily increasing the permeability of the membranes 54 of local cells of the olfactory region, increasing the diffusion of the therapeutic compound into the cells 52 and onto the receptor sites, allowing for decreased total body dosages, decreased side effects and enabling new therapies.

The pharmaceutical agent may include, for example, but not limited to cannabinoids and terpenes in certain ratios in appropriate vehicles. The micronized pharmaceutical agent liquid has an effect of temporarily permeating the olfactory membranes 54 of the local olfactory cells 52 of the olfactory pathway, thereby increasing diffusion of the therapeutic compounds into the cells that pass through the blood brain barrier for immediate effect. This allows allowing for decreased total body dosages, decreased side effects and enabling new therapies. This method does not require combustion and is therefore addresses one of the problems and unmet needs found with other combustible/smoking and vapor which are common delivery methods of medicinal cannabis. The method avoids lung involvement, which is important for those patients in treatment for lung cancer, COPD and emphysema. It provides a faster method of onset of relief than the oral route of administration using extracts or infused foods.

The non-intoxicating cannabinoids- a class of diverse chemical compounds that act on cannabinoid receptors and vallinoid receptors in cells found in the body and in the nasal cavity where the olfactory system resides, alter the neurotransmitter release in the brain. Ligands for these receptor proteins include the endocannabinoids, the phytocannabinoids, and synthetic cannabinoids. The most notable cannabinoid is the phytocannabinoid tetrahydrocannabinol, the primary psychoactive compound in cannabis. Cannabidiol is another major constituent of the plant i.e. cannabidiol (CBD) as one example along with the other known and identified to date but not excluding any additional phytocannabinoids that may be identified in the future to include the acid versions which are the precursors and the decarboxylated versions as well to include CBDA, CBD, CBGA, CBG, CBNA, CBN, THCA. The psychoactive phytocannabinoid delta-9-THC, tetrahydrocannabinol have great potential for the treatment of anxiety, stress, insomnia, atopic dermatitis skin disorders such as eczema and pain related to neuralgia, migraine and other disease states.

Nasal delivery of pharmaceutical agents such as the cannabinoids bypasses the lung and the liver and reaches the specific receptors that mediate pain and inflammation directly. While chronic pain relief may be best achieved through a transdermal route, ‘breakthrough’ pain in be best alleviated with intranasal (IN) delivery and olfactory delivery (OFD). Combining IN /OFD of the present device and transdermal delivery for phytocannabinoids may provide patients with a needs-driven treatment in the form of a non-addictive non-opioid therapy.

The formulations of the pharmaceutical agent in liquid are generally hydrophilic, but a lipophilic pharmaceutical agent at the low dosages envisioned will be sufficiently soluble in hydrophilic liquids to form aqueous solutions by sonication. Formulations includes mixtures of non-volatile and volatile ingredients used as either permeability enhancers or solubilizers or adjunct ingredients to facilitate bioavailability. These ingredients can come from a wide class of compounds known to those of ordinary skill in the art as adjuncts, solubilizers, co-solubilizers and emulsifiers.

Formulations may include but not be limited to a wide variety of adjuncts including polysorbates, polyethylene glycol, cyclodextrin, beta-cyclodextrin, cholesterol, lecithin, natural extract of Quillaia saponin, phospholipids, such as phosphatidylcholine, as drug delivery carriers.

In one example embodiment, a formulation includes phytocannabinoid compositions.

In another example embodiment, a formulation includes phytocannabinoid compositions with terpenoids and co-solubilizers.

In another example embodiment, a formulation includes phytocannabinoid compositions with terpenoids, flavinoids and essential oils, vitamins, herbal extracts, adjuncts, solubilizers, co-solubilizers and emulsifiers.

A typical concentrate will contain the active ingredient (e.g. phytocannabinoid oil/cannabis oil extract), cyclodextrin, terpene (such as beta-caryophyllene, alpha-pinene, limonene, linalool as non-limiting examples), carrier oil (a high oleic acid vegetable oil such as olive oil and hempseed oil as non-limiting examples), surfactant (Quillaia saponin as a non-limiting example) and water.

A typical concentrate formulation is presented in Table 1.

TABLE 1

| Ingredient | Typical Concentration |
|---|---|
| Active moiety | 5.0% |
| Terpene | 1.2% |
| Carrier oil | 7.0% |
| Surfactant | 2.0% |
| Water | 84.8% |

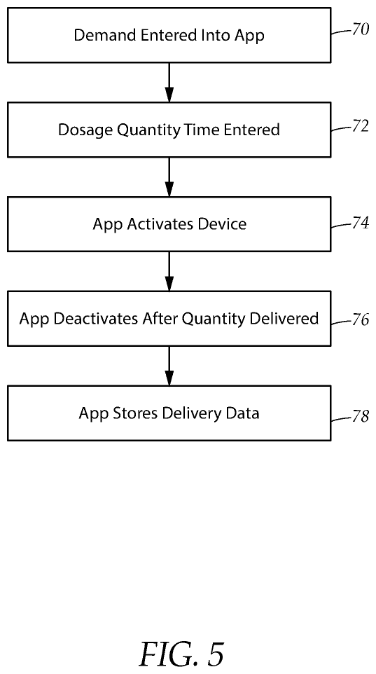

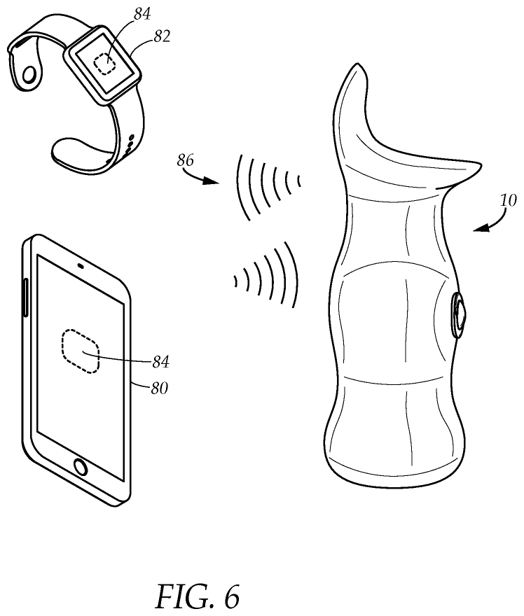

Referring to FIGS. 5 and 6, the device and method can include a software application or app 84 that runs on a variety of mobile computing platforms such as a smartphone, a smart watch, a tablet computer or other hybrid mobile computing platforms. FIG. 6 displays how a smart watch 82 or smartphone 80 communicates wirelessly 86 over a wireless network with the device 10.

The app 84 provides an on-demand activation for dosing delivery. The app 84 is programmed with a schedule the dosage, quantity and time of activation, computes the number of doses delivered and calculates the dosage delivered and metered by the device. This provides to the patient knowledge of the minimum effective dose resulting in better patient compliance. The minimum effective dose knowledge is particularly useful who are in treatment for pain management and the side effects of chemotherapy.

To initiate therapy, the patient enters a demand into the app 70. This demand may be activated by displaying a QR code or bar code on a patient's identification tag, the mobile computing device reading and identifying the patient. The schedule of dosage, quantity and time as well as duration are either retrieved from storage or from the QR code 72. The app, using a wireless network as explained hereinabove activates the device 74. Once the device delivers the quantity of the treatment and defined in step 72, the app deactivates the device 76. The app stores the delivery data 78 either locally in the mobile computing device or on a server. The server may be local or cloud-based.

It is understood that when an element is referred hereinabove as being “on” another element, it can be directly on the other element or intervening elements may be present therebetween. In contrast, when an element is referred to as being “directly on” another element, there are no intervening elements present.

Moreover, any components or materials can be formed from a same, structurally continuous piece or separately fabricated and connected.

It is further understood that, although ordinal terms, such as, “first,” “second,” “third,” are used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

Spatially relative terms, such as “beneath,” “below,” “lower,” “above,” “upper” and the like, are used herein for ease of description to describe one element or feature's relationship to another element(s) or feature(s) as illustrated in the figures. It is understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

Example embodiments are described herein with reference to cross section illustrations that are schematic illustrations of idealized embodiments. As such, variations from the shapes of the illustrations as a result, for example, of manufacturing techniques and/or tolerances, are to be expected. Thus, example embodiments described herein should not be construed as limited to the particular shapes of regions as illustrated herein, but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

The disclosed example embodiments may individually and/or collectively be components of a larger system, wherein other procedures may take precedence over or otherwise modify their application. Also, a number of steps may be required before, after, and/or concurrently with the following embodiments.

As will be appreciated by one skilled in the art, aspects of the present disclosure may be embodied as a system, method or software application. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.”

Aspects of the present disclosure are described below with reference to network illustrations and/or block diagrams of methods, systems and software applications according to embodiments of the disclosure. Each block of the block diagram, and combinations of blocks in the block diagrams, can be implemented by software application instructions. These software application instructions may be provided to a processor of a general purpose handheld computing device, special purpose handheld computing device, or other programmable handheld computing apparatus to produce a machine, such that the instructions, which execute via the processor of the handheld computing device or other programmable data processing apparatus, create means for implementing the functions/acts specified in the network and/or block diagram block or blocks.

The network and block diagrams in the Figures illustrate the architecture, functionality and operation of possible implementations of systems, methods and software applications according to various embodiments of the present disclosure. In this regard, each block in the network or block diagrams may represent a module, segment or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. Each block of the block diagrams and/or network illustration, and combinations of blocks in the block diagrams and/or network illustration, can be implemented by special purpose hardware-based systems that perform the specified functions or acts, or combinations of special purpose hardware and software instructions.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

The network and block diagrams depicted herein are just one example. There may be many variations to this diagram or the steps (or operations) described therein without departing from the spirit of the disclosure. For instance, the steps may be performed in a differing order and/or steps may be added, deleted and/or modified. All of these variations are considered a part of the claimed disclosure.

While the preferred embodiment to the disclosure had been described, those skilled in the art, both now and in the future, may make various improvements and/or enhancements which fall within the scope of the claims which follow. These claims should be construed to maintain the proper protection for the disclosure first described.

In conclusion, herein is presented a device and an olfactory delivery device and method for delivery of a variety of pharmaceutical agents. The disclosure is illustrated by example in the drawing figures, and throughout the written description. It should be understood that numerous variations are possible, while adhering to the inventive concept. Such variations are contemplated as being a part of the present disclosure.

What is claimed is:

1. A method for administering a pharmaceutical agent, comprising:
preparing a microfluidized liquid containing a pharmaceutical agent by dissolving the pharmaceutical agent in an aqueous solution containing a terpene, a surfactant and a carrier oil, the pharmaceutical agent solubilizing by sonication cavitation, forming liquid particles less than 5 microns in size; and
delivering the microfluidized liquid containing the pharmaceutical agent into an olfactory region of a nasal cavity by inhaling the microfluidized liquid through a nose, the pharmaceutical agent binding to a plurality of receptor cells with an olfactory membrane, transmitting across a plurality of neurons of an olfactory nerve in an olfactory bulb, leading directly to a brain.

2. The method as described in claim 1, wherein the step of preparing a microfluidized liquid containing a pharmaceutical agent by dissolving the pharmaceutical agent in the aqueous solution includes a lipophilic pharmaceutical agent.

3. The method as described in claim 2, wherein the step of delivering the microfluidized liquid containing the pharmaceutical agent into the olfactory region of the nasal cavity by inhaling the microfluidized liquid through the nose, the pharmaceutical agent binding to the plurality that includes cannabinoid and vallinoid receptors.

4. The method as described in claim 3, wherein the step of delivering the microfluidized liquid containing the pharmaceutical agent into the olfactory region of a nasal cavity by inhaling the microfluidized liquid containing lipids through the nose, increases bioavailability of a lipophilic pharmaceutical agent.

5. The method as described in claim 4, wherein delivering the microfluidized liquid containing the pharmaceutical agent into the olfactory region of a nasal cavity directly by inhaling the microfluidized liquid through the nose, the pharmaceutical agent delivered directly to a plurality of receptor sites in the brain thereby bypassing and protecting a pair of lungs and avoiding a first-pass metabolises in a liver.
